(19) United States
(12) Patent Application Publication
(10) Pub. No.: US 2023/0326595 A1
(43) Pub. Date: Oct. 12, 2023

(54) E-HEALTH INSIGHTS OF WHOLE BODY VIBRATION HEALTH MONITORING

(71) Applicant: Kyndryl, Inc., New York, NY (US)

(72) Inventors: Prasanna Kumar Dixit J, Bangalore (IN); Sharat K. Tripathy, Bangalore (IN)

(21) Appl. No.: 17/716,664

(22) Filed: Apr. 8, 2022

(51) Int. Cl. G16H 50/20; G16H 40/63

(52) U.S. Cl. CPC G16H 50/20; G16H 40/63

(57) ABSTRACT

Aspects of the present disclosure relate generally to e-health insights of health monitoring of vibration impacts on the human body and, more particularly, to health monitoring of whole body vibration by the Internet of Things. For example, a computer-implemented method includes inputting into a health model, by the computing device, a plurality of health data and sensor data collected for an individual for a predetermined time period; determining, by the computing device, a probability of an onset of at least one symptom of whole body vibration syndrome for the individual from the plurality of health data and sensor data; and sending, by the computing device, a prediction of the onset of the at least one symptom of whole body vibration syndrome to a user device for display on the user device.

### BACKGROUND

Aspects of the present invention relate generally to e-health insights of health monitoring of vibration impacts on the human body and, more particularly, to health monitoring of whole body vibration by the Internet of Things.

Millions of workers are exposed to mechanical vibration transmitted to their hands from powered tools or transmitted to their whole body from sitting in industrial vehicles. Road roughness is one example of a source of vibration in vehicles, and such vibration, in turn, exposes drivers and passengers to whole-body vibration (WBV). Regulatory legislation prescribes limits of allowable vibration at work for health and safety concerns.

### SUMMARY

In a first aspect of the invention, there is a computer-implemented method including: inputting into a health model, by the computing device, a plurality of health data and sensor data collected for an individual for a predetermined time period; determining, by the computing device, a probability of an onset of at least one symptom of whole body vibration syndrome for the individual from the plurality of health data and sensor data; and sending, by the computing device, a prediction of the onset of the at least one symptom of whole body vibration syndrome to a user device for display on the user device.

In another aspect of the invention, there is a computer program product including one or more computer readable storage media having program instructions collectively stored on the one or more computer readable storage media. The program instructions are executable to: receive, by a computing device, a collection of health data and sensor data for a plurality of individuals for a predetermined period of time; train, by the computing device, a health model from the collection of the health data and sensor data; store, by the computing device, the trained health model on the one or more computer readable storage media; and determine, by the computing device, a probability of the onset of at least one symptom of whole body vibration syndrome for an individual from the trained health model.

In another aspect of the invention, there is a system including a processor, a computer readable memory, one or more computer readable storage media, and program instructions collectively stored on the one or more computer readable storage media, the program instructions executable to: receive, by a computing device, a plurality of health data and sensor data collected for an individual for a predetermined time period as a plurality of parameters for input into a health model to predict an onset of at least one symptom of whole body vibration syndrome for the individual; apply, by the computing device, a time-to-event analysis of the health model to analyze the plurality of health data and the sensor data collected for the individual for the predetermined time period to determine the probability of the onset of symptoms of whole body vibration syndrome; determine, by the computing device, a probability of the onset of the at least one symptom of whole body vibration syndrome estimated at time t by a quotient of a plurality of individuals used to train the health model who did not develop any symptom of whole body vibration syndrome beyond time t divided by the total number of the plurality of individuals; and send, by the computing device, a prediction of the onset of the at least one symptom of whole body vibration syndrome to a user device for display on the user device.

### BRIEF DESCRIPTION OF THE DRAWINGS

Aspects of the present invention are described in the detailed description which follows, in reference to the noted plurality of drawings by way of non-limiting examples of exemplary embodiments of the present invention.

FIG. 1 depicts a cloud computing node according to an embodiment of the present invention.

FIG. 2 depicts a cloud computing environment in accordance with aspects of the invention.

FIG. 3 depicts abstraction model layers in accordance with aspects of the invention.

FIG. 4 shows a block diagram of an exemplary environment in accordance with aspects of the invention.

FIG. 5 shows a flowchart of an exemplary method in accordance with aspects of the invention.

FIG. 6 shows a flowchart of an exemplary method in accordance with aspects of the invention.

### DETAILED DESCRIPTION

Aspects of the present invention relate generally to e-health insights of health monitoring of vibration impacts on the human body and, more particularly, to health monitoring of whole body vibration by the Internet of Things (IoT). More specifically, aspects of the invention relate to methods and systems for providing e-health insights on potential risks of forces impacting worker health derived using a collective perception obtained from IoT. For example, the methods, systems and program products described herein support services for health monitoring of whole body vibration from sensor data provided by IoT sensor devices to predict symptomatic whole body vibration which may impact workers. According to aspects of the invention, the methods, systems and program products described herein receive health and practice data of a user and sensor data of whole body vibration of a user to predict the onset of symptomatic whole body vibration syndrome.

In embodiments, the methods, systems and program products described herein receive health data and associated sensor data collected for an individual for a length of time and input this data as parameters into a health model to predict the onset of whole body vibration syndrome for the individual. The health data, for instance, may include age, comorbidity information, health issue information and the time to symptoms of a health issue, as well as practices of the individual such as smoking, sleeping and exercise practices. The associated sensor data collected for the individual, for instance, may include accelerometer information measuring the intensity of vibration, the duration of vibration from a start time until an end time, in addition to sensors which sense and/or measure average temperature during the duration of vibration, and average acoustic noise during the duration of vibration, amongst other items. This sensor data may be provided by IoT sensor devices including, without limitation, an accelerometer device to sense vibration, an instrument to measure the level of vibration, a microphone to detect acoustic noise information, a temperature sensor, and a camera to capture images of object causing air pollution. The health model provides a probability of the onset of symptomatic whole body vibration syndrome for the individual from the data and the associated sensor data collected for the individual for the length of time, and a prediction when a symptom will appear in an individual can be sent for display on the individual's user device. This prediction may be communicated to the individual for consideration to mitigate the risks of whole body vibration syndrome in their occupation and may additionally serve to diagnose vibration-induced disorders at an early stage in their development or, even, prior to development.

Aspects of the present invention are directed to improvements in computer-related technology. In embodiments, the system including a processor, a computer readable memory, one or more computer readable storage media, and program instructions collectively stored on the one or more computer readable storage media may receive a plurality of data and associated sensor data collected for an individual for input into a health model to predict an onset of at least one symptom of whole body vibration syndrome or the probability of the onset of symptoms of whole body vibration syndrome. The results may be sent as a presentation to a user device for display on the user device. These are specific improvements in the way computers may operate and interoperate to detect and to diagnose vibration-induced disorders. Implementations of the invention describe additional elements that are specific improvements in the way computers may operate and these additional elements provide non-abstract improvements to computer functionality and capabilities. As an example, a computer program product including one or more computer readable storage media having program instructions collectively stored on the one or more computer readable storage media may receive, by a computing device, a collection of data and associated sensor data which is used to train the health model to predict an onset of at least one symptom of whole body vibration syndrome for an individual.

It should be understood that, to the extent implementations of the invention collect, store, or employ personal information provided by, or obtained from, individuals, such information shall be used in accordance with all applicable laws concerning protection of personal information. Additionally, the collection, storage, and use of such information may be subject to consent of the individual to such activity, for example, through “opt-in” or “opt-out” processes as may be appropriate for the situation and type of information. Storage and use of personal information may be in an appropriately secure manner reflective of the type of information, for example, through various encryption and anonymization techniques for particularly sensitive information.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium or media, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

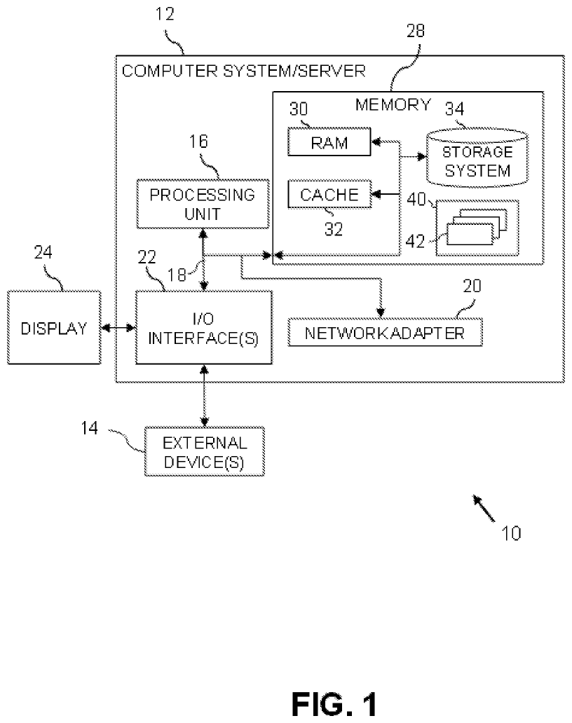

Referring now to FIG. 1, a schematic of an example of a cloud computing node is shown. Cloud computing node 10 is only one example of a suitable cloud computing node and is not intended to suggest any limitation as to the scope of use or functionality of embodiments of the invention described herein. Regardless, cloud computing node 10 is capable of being implemented and/or performing any of the functionality set forth hereinabove.

In cloud computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 1, computer system/server 12 in cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

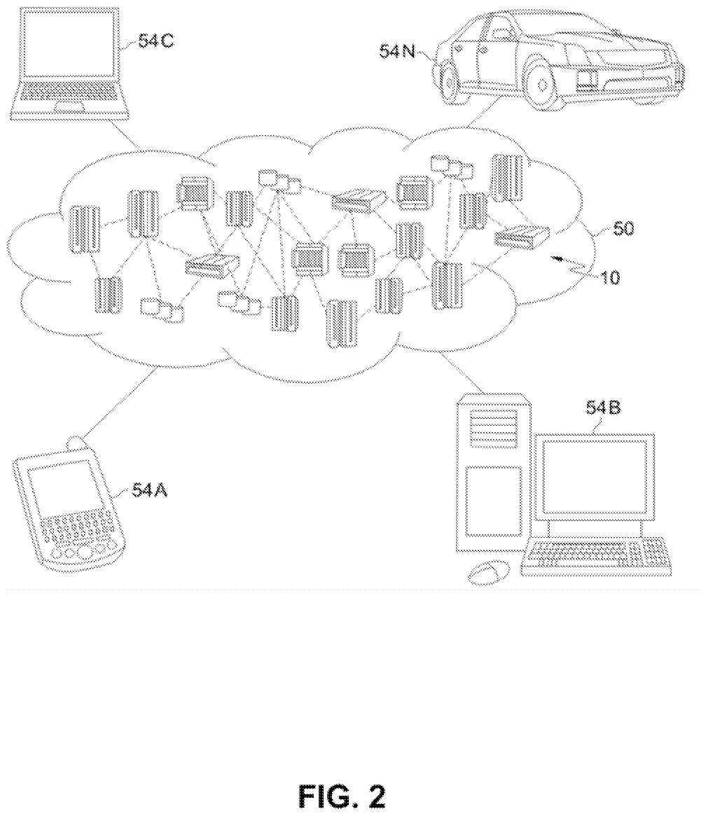

Referring now to FIG. 2, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 2 are intended to be illustrative only and that computing nodes 10 and cloud computing environment 50 can communicate with any type of computerized device over any type of network and/or network addressable connection (e.g., using a web browser).

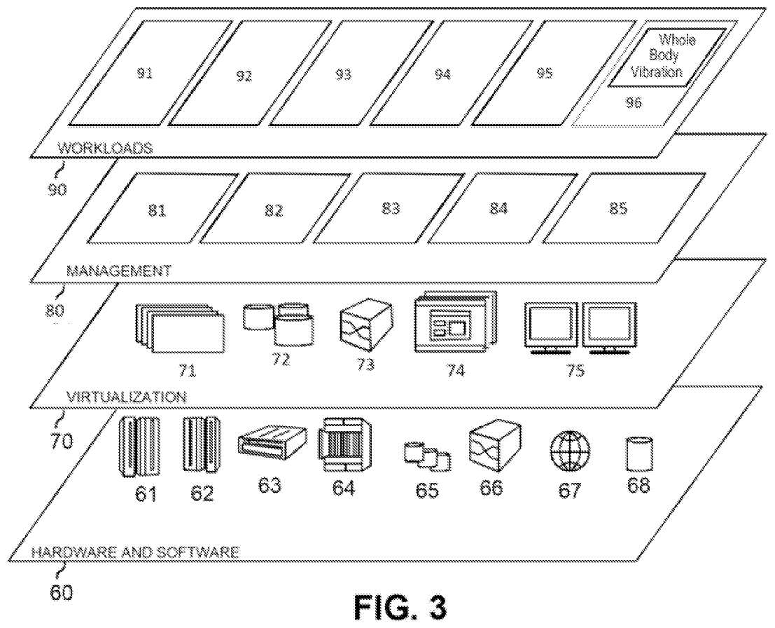

Referring now to FIG. 3, a set of functional abstraction layers provided by cloud computing environment 50 (FIG. 2) is shown. It should be understood in advance that the components, layers, and functions shown in FIG. 3 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below.

Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and whole body vibration processing 96.

Implementations of the invention may include a computer system/server 12 of FIG. 1 in which one or more of the program modules 42 are configured to perform (or cause the computer system/server 12 to perform) one of more functions of the rapid data replication processing 96 of FIG. 3. For example, the one or more of the program modules 42 may be configured to receive health data and associated sensor data collected for an individual for a length of time and input this data as parameters into a health model to predict the onset of whole body vibration syndrome for the individual as further described herein in more detail.

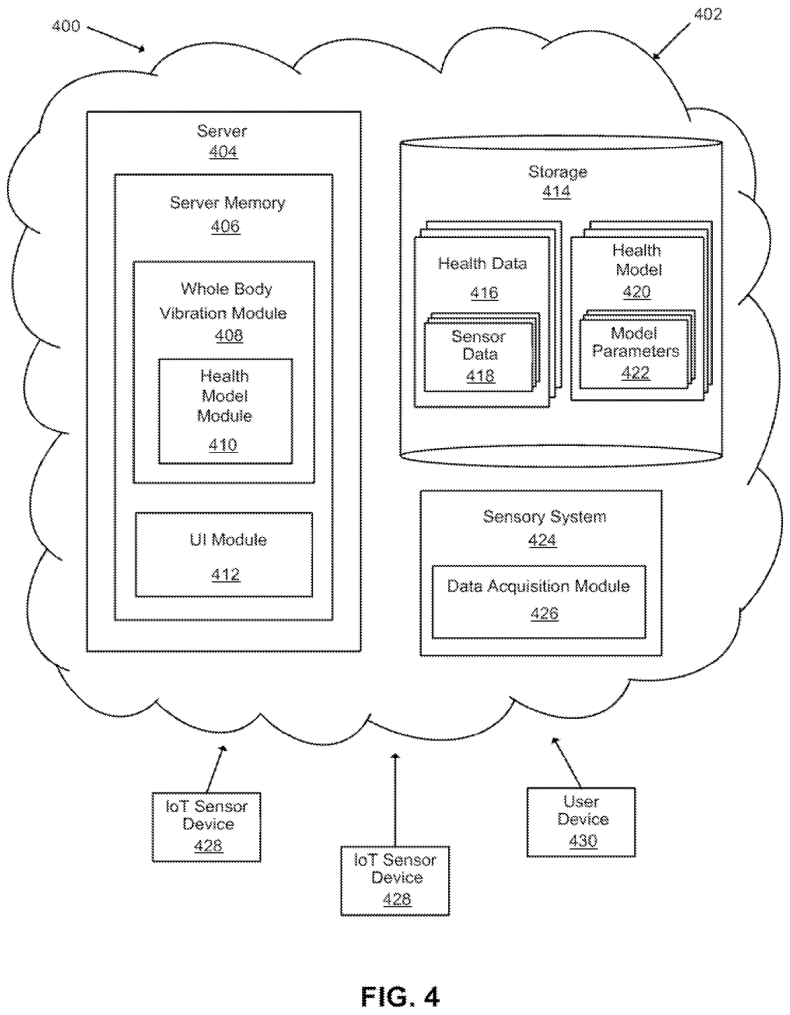

FIG. 4 shows a block diagram of a server in a cloud computing environment in accordance with aspects of the present invention. In embodiments, the cloud computing environment 400 includes a server 404, which may be a computer system such as a computer system 12 described with respect to FIG. 1 and is a cloud computing node such as cloud computing node 10 described with respect to FIG. 2 with which computing devices used by cloud consumers may communicate over a network 402. In general, the server 404 supports services for health monitoring of whole body vibration from sensor data provided by IoT sensor devices and for prediction of symptomatic whole body vibration which may impact the health of users.

The server 404 has a server memory 406 such as memory 28 described with respect to FIG. 1. The server 404 includes, in memory 406, a whole body vibration module 408 having functionality to receive health and practice data of a user and sensor data of whole body vibration of the user to predict the onset of symptomatic whole body vibration syndrome. The server 404 also includes, in memory 406, a user interface (UI) module 412 having functionality to provide a presentation of a prediction of the onset of symptomatic whole body vibration syndrome for display on a user device 430. The whole body vibration module 408 includes a health model module 410 having functionality to generate a health model to predict the onset of symptomatic whole body vibration syndrome from a collection of health and practice data of users and sensor data of whole body vibration of the users, and to apply the health model to predict the onset of symptomatic whole body vibration syndrome of the individual user.

In embodiments, the whole body vibration module 408, health model module 410, the user interface module 412, and the data acquisition module 426 may comprise one or more program modules such as program modules 42 described with respect to FIG. 1. The server 404 may include additional or fewer modules than those shown in FIG. 4. In embodiments, separate modules may be integrated into a single module. Additionally, or alternatively, a single module may be implemented as multiple modules. Moreover, the quantity of devices and/or networks in the environment is not limited to what is shown in FIG. 4. In practice, the environment may include additional devices and/or networks; fewer devices and/or networks; different devices and/or networks; or differently arranged devices and/or networks than illustrated in FIG. 4.

In accordance with aspects of the invention, FIG. 4 also shows a block diagram of storage 414 in a cloud computing environment. In embodiments, the cloud computing environment 400 may be a computer system such as a computer system 12 described with respect to FIG. 1 having computer storage such as storage system 34 also described with respect to FIG. 1, and may be a cloud computing node such as cloud computing node 10 described with respect to FIG. 2 with which computing devices used by cloud consumers may communicate over a network 402. In general, the storage 414 may store health data in files 416 for users and includes information of sensor data 418 of whole body vibration of a user collected over a period of time. For example, the health data may include age, comorbidity information, health issue information and the time to symptoms of a health issue, and so forth of the user, as well as practices of the user such as smoking, sleeping and exercise practices to name a few. The sensor data 418 may include accelerometer information measuring the intensity of vibration, the duration of vibration from a start time until an end time, in addition to the average temperature during the duration of vibration, and the average acoustic noise during the duration of vibration, and so forth. Those skilled in the art should appreciate that other sensor data may be used including, for example, the source of the vibration such as from drilling, driving, or operating other equipment producing vibration.

The storage 414 may also store a health model 420 that includes model parameters 422 used to generate the health model 420. For example, the health model 420 may predict time_to_symptom (S) of the onset of symptomatic whole body vibration syndrome for a user from a number of environmental conditions experienced by the user including vibration exposure (V), ambient temperature (T) and acoustic (A) noise pollution and from a number of health conditions and practices of the user such as age, comorbidity information, health issue information and the time to symptoms of the health issue and personal practices as illustrative non-limiting examples. These environmental conditions, health conditions and practices represent model parameters 422 used to generate the health model 420 to predict the time_to_symptom (S) of the onset of symptomatic whole body vibration syndrome for the user.

In accordance with aspects of the invention, FIG. 4 also shows a block diagram of a sensory system 424 in a cloud computing environment 400 in accordance with aspects of the present invention. In embodiments, the cloud computing environment 400 and sensory system 424 may be a computer system such as a computer system 12 described with respect to FIG. 1 and a cloud computing node such as cloud computing node 10 described with respect to FIG. 2 with which computing devices such as IoT sensor devices 428 and a user device 430 may communicate over a network 402. In general, the sensory system 424 supports services for receiving and storing sensor data, for instance as sensor data 418, provided by IoT sensor devices 428 for health monitoring of whole body vibration.

The sensory system 424 includes a data acquisition module 426 having functionality to receive sensor data of whole body vibration of a user. For example, the data acquisition module may receive sensor data from IoT sensor devices such as vehicle instrumentation measuring shock and vibration at locations within a vehicle. Such instrumentation for measuring shock and vibration may include, for example, instrumentation having an accelerometer device to sense the vibration and an instrument to measure the level of vibration, or a seat accelerometer, or other kinds of accelerometers such as an accelerometer on wearable devices. The data acquisition module 426 may also receive acoustic noise information from an IoT sensor device having a microphone, ambient temperature information from an IoT sensor device having a temperature sensor, and pollution information from a IoT device having a high resolution megapixel reflex camera that may capture images of vehicular density or other pollution causing objects. The vibration and shock information, the acoustic noise information, the ambient temperature information and the pollution information are environmental conditions that can be used as model parameters 422 to generate the health model 420 to predict the time_to_symptom(S) of the onset of symptomatic whole body vibration syndrome for the user.

In accordance with aspects of the invention, FIG. 4 also shows user device 430 that communicates with server 404 and additionally shows Internet of Things (IoT) devices 428 that communicate with sensory system 424 in the cloud computing environment 400. The server 404 supports services for health monitoring of whole body vibration from sensor data provided by IoT sensor devices 428 used to generate a health model and used to predict symptomatic whole body vibration impacting the health of users. In embodiments, the user device 430 and IoT devices 428 may be smart phones, tablets, computers, wireless instrumentation or sensors devices, or any electronic device that can detect information in its environment and communicate the information over a network in a cloud computing environment. Examples of such instrumentation, sensors and devices include, without limitation, an accelerometer device to sense vibration, an instrument to measure the level of vibration, a microphone to detect acoustic noise information, a temperature sensor, and a camera to capture images of object causing air pollution.

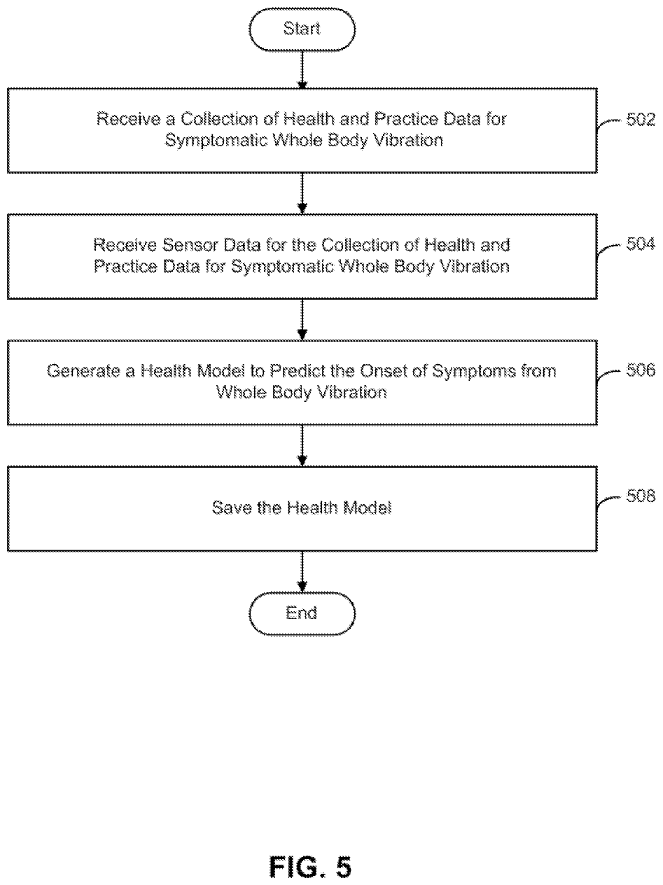

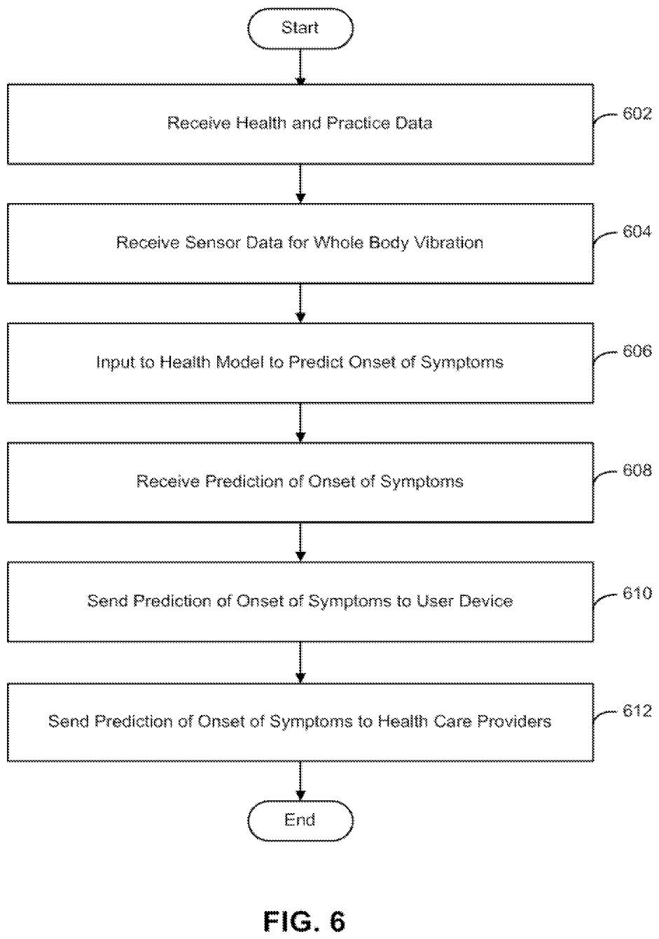

FIGS. 5-6 show flowcharts and/or block diagrams that illustrate the architecture, functionality, and operation of possible implementations of systems, methods, and computer program products according to various embodiments of the present invention. As noted above, each block may represent a module, segment, or portion of instructions, which comprises one or more executable instructions for implementing the specified logical function(s). The functions noted in the blocks may occur out of the order, two blocks shown in succession may, in fact, be accomplished as one step, executed concurrently, substantially concurrently, in a partially or wholly temporally overlapping manner, or the blocks may sometimes be executed in the reverse order, depending upon the functionality involved. And some blocks shown may be executed and other blocks not executed, depending upon the functionality involved.

FIG. 5 shows a flowchart of an exemplary method in accordance with aspects of the present invention. Steps of the method may be carried out in the environment of FIG. 4 and are described with reference to elements depicted in FIG. 4. In particular, the flowchart of FIG. 5 shows an exemplary method for generating a health model in a cloud computing environment, in accordance with aspects of the present invention.

At step 502, the system receives a collection of health and practice data with factors for individuals that are input as model parameters for generating a health model to predict symptomatic whole body vibration in an embodiment. Among the factors of health are age, other comorbidities, and time to symptom (time from starting the occupation to symptom of health issue). There may be time to symptom information for some individuals in the collection of health and practice data, and there will not be for others who have not yet developed any symptom since the time they started the occupation or they have left the occupation before any symptom ever developed. For example, whole body vibration module 408 implemented on server 404 may receive a collection of health and practice data from health data files 416 in storage 414, with factors for individuals that are input as model parameters for generating the health model to predict symptomatic whole body vibration.

At step 504, the system receives sensor data for the collection of health and practice data with parameters for individuals that are input as model parameters for generating a health model to predict symptomatic whole body vibration in an embodiment. Among the parameters of the sensor data are the vibration exposure, ambient temperature, acoustic noise pollution, and air pollution, etc. In embodiments, vibration is one factor influencing the health hazard of whole body vibration syndrome. Accordingly, the intensity and duration of vibration exposure is collected from sensors, such as an accelerometer, for an individual on a regular basis. Heat exposure in the surrounding environment is also a factor and may be generated by running engines or other equipment operating within close proximity of an individual. Additionally, noise caused by other machines or crowds in proximity can be measured. And cameras can capture images of nearby objects causing air pollution to give an estimation on the density of nearby polluting objects which can have a major influence on the health risk. For example, whole body vibration module 408 implemented on server 404 may receive sensor data for the collection of health and practice data, from health data files 416 in storage 414, with parameters for individuals that are input as model parameters for generating the health model to predict symptomatic whole body vibration.

At step 506, the system generates a health model to predict the onset of symptoms from whole body vibration. The health analysis, or more generally, time-to-event analysis, employs to a set of methods for analyzing the length of time until the occurrence of an event of interest, and in particular, the time to the onset of symptoms of whole body vibration syndrome. The health model is trained with multivariate data of the input parameters, namely the collection of health and practice data and associated sensor data, and the trained model provides a probability of time to symptom for a given sample of input parameters of an individual. Thus, the model may provide a prediction for an individual from the data collected for the parameters for the individual in advance of any symptom of whole body vibration syndrome.

The health model applies a health or time-to-event analysis to generate an initial model which may be represented by the function S(t)=P(T>t), where T is any random variable denoting time before any symptom develops in an individual. Thus, this health function gives the probability of not developing any symptom beyond time t. The health function at time t can be estimated by the ratio of candidates not developing any symptom beyond time t and the total number of candidates as follows: S(t)=(number of candidates with no symptom beyond t)/(total number of candidates). Accordingly, the health model can provide the probability that an individual will get a symptom, and, if so, when the first symptom may appear.

A feature of health data is that typically not all individuals experience the event by the end of an observation period, so the actual health times or occurrences of the event for some individuals may be unknown. This phenomenon, referred to as censoring, is accounted for in the analysis to allow for valid inferences. Thus, health analysis can be applied in a range of situations such as predicting the onset of whole body vibrations syndrome in which time to symptom is analyzed in terms of its occurrence or non-occurrence during a specified observation period. In embodiments, the health analysis may be performed using the Kaplan-Meier method or Cox proportional hazards regression modeling that supports censoring, given that many individuals have not yet developed any symptoms since the time they joined an occupation, or they have left the occupation before they experienced any symptoms of whole body vibration syndrome.

The model may also output a ranked list of covariates that influence development of a symptom from whole body vibration. In an embodiment, the vibration duration is one influential factor in the model for the time to symptom, and other factors include vibration intensity, acoustic noise, comorbidity and other practices. In embodiments, whole body vibration module 408 implemented on server 404 may generate the health model to predict the onset of symptoms from whole body vibration from the collection of health and practice data with the associated sensor data stored in health data files 416 in storage 414.

At step 508, the system saves the health model in computer storage. For example, whole body vibration module 408 implemented on server 404 may save the health model in the health model file 420 in storage 414. The trained health model can be used to provide the probability that an individual will get a symptom for a given sample of input parameters of an individual, and, if so, when the first symptom will appear. Thus, the model may provide a prediction for an individual from the data collected for the parameters for the individual in advance of any symptom of whole body vibration syndrome. This prediction may be communicated to the individual for consideration to mitigate the risks for the occupational hazard.

FIG. 6 shows a flowchart of an exemplary method in accordance with aspects of the present invention. Steps of the method may be carried out in the environment of FIG. 4 and are described with reference to elements depicted in FIG. 4. In particular, the flowchart of FIG. 6 shows an exemplary method for predicting the onset of symptoms from whole body vibration syndrome for an individual from the data of health and practices of the individual with the associated sensor data in a cloud computing environment, in accordance with aspects of the present invention.

At step 602, the system receives health and practice data with factors for an individual that are input as parameters to the health model that predicts symptomatic whole body vibration. Among the factors of health are age, other comorbidities, and time to symptom (time from starting the occupation to symptom of first health issue). There may be time to symptom information for an individual in the health and practice data, and there may not be in the case of an individual who has not yet developed any symptom since the time that the individual started the occupation or who has left the occupation before any symptom ever developed. In embodiments, whole body vibration module 408 implemented on server 404 may receive the health and practice data from health data files 416 in storage 414, with factors for the individual that are input as parameters to the health model 420 that predicts symptomatic whole body vibration.

At step 604, the system receives sensor data for the health and practice data with parameters for an individual that are input as model parameters to the health model that predicts symptomatic whole body vibration. Among the parameters of the sensor data are the vibration exposure, ambient temperature, acoustic noise pollution, and air pollution. In embodiments, vibration is one of the crucial factors influencing the health hazard of whole body vibration syndrome. Accordingly, the intensity and duration of vibration exposure is collected from sensors, such as an accelerometer, for an individual on a regular basis. Heat exposure in the surrounding environment is also a factor and may be generated by running engines or other equipment operating within close proximity of an individual. Additionally, noise caused by other machines or crowds in proximity can be measured. And cameras can capture images of nearby objects causing air pollution to give an estimation on the density of nearby polluting objects which can have an influence on the health risk. In embodiments, the whole body vibration module 408 implemented on server 404 may receive sensor data for the health and practice data, from health data files 416 in storage 414, with parameters for individuals that are input as model parameters to the health model that predicts symptomatic whole body vibration.

At step 606, the system inputs the health and practice data and the associated sensor data to a health model that predicts the onset of symptomatic whole body vibration in an embodiment. The health model is trained with the multivariate data of input parameters from the collection of health and practice data and associated sensor data, and the trained model provides a probability of time to symptom for a given sample of input parameters of an individual. The health model applies a health or time-to-event analysis to generate an initial model which may be represented in an embodiment by the function S(t)=P(T>t), where T is any random variable denoting time before any symptom develops in an individual. Thus this health function gives the probability of not developing any symptom beyond time t. The health function at time t can be estimated by the ratio of candidates not developing any symptom beyond time t and the total number of candidates as follows: S(t)=(number of candidates with no symptom beyond t)/(total number of candidates). Accordingly, the health model can provide the probability that an individual will get a symptom, and, if so, when the first symptom will appear. In embodiments, the whole body vibration module 408 implemented on server 404 may input the health and practice data and the associated sensor data to the health model that predicts the onset of symptomatic whole body vibration.

At step 608, the system receives a prediction of the onset of symptoms of whole body vibration for an individual from the health analysis of the input parameters of the health and practice data and the associated sensor data for the individual collected over a length of time. For example, the health model can provide the probability that an individual will get a symptom of whole body vibration syndrome, and, if so, when the first symptom will appear. In embodiments, the whole body vibration module 408 implemented on the server 404 may receive a prediction of the onset of symptoms of whole body vibration for an individual from the health analysis of the input parameters of the health and practice data and the associated sensor data for the individual collected over a length of time.

And at step 610, the system sends the prediction of the onset of symptoms of whole body vibration syndrome for an individual to a user device. This prediction may be communicated to the individual before the onset of symptoms for consideration to mitigate the risks of the occurrence of whole body vibration syndrome. For example, a presentation of the prediction may be sent to the user device of an individual indicating when the first symptom will appear given the health and practice data and associated sensor data of the individual. In an embodiment, a message may accompany the prediction that recommends the user change the route of travel by the user. In embodiments, user interface (UI) module 412 implemented on server 404 may send a presentation of the prediction of the onset of symptomatic whole body vibration syndrome for display on user device 430. And at step 612, the system may also send the prediction of the onset of symptoms of whole body vibration syndrome for an individual to a user device of a health care provider.

In this way, aspects of the present invention may support services for health monitoring of whole body vibration from sensor data provided by IoT sensor devices that may be analyzed by a health model to predict symptomatic whole body vibration impacting the health of workers. Those skilled in the art should appreciate that other sensor data of whole body vibration of a user may be used in embodiments including, for example, the source of the vibration such as from drilling, driving, or operating other equipment producing vibration.

In embodiments, a service provider could offer to perform the processes described herein. In this case, the service provider can create, maintain, deploy, support, etc., the computer infrastructure that performs the process steps of the invention for one or more customers. These customers may be, for example, any business that uses technology. In return, the service provider can receive payment from the customer(s) under a subscription and/or fee agreement and/or the service provider can receive payment from the sale of advertising content to one or more third parties.

In still additional embodiments, the invention provides a computer-implemented method, via a network. In this case, a computer infrastructure, such as computer system 12 (FIG. 1), can be provided and one or more systems for performing the processes of the invention can be obtained (e.g., created, purchased, used, modified, etc.) and deployed to the computer infrastructure. To this extent, the deployment of a system can comprise one or more of: (1) installing program code on a computing device, such as computer system 12 (as shown in FIG. 1), from a computer-readable medium; (2) adding one or more computing devices to the computer infrastructure; and (3) incorporating and/or modifying one or more existing systems of the computer infrastructure to enable the computer infrastructure to perform the processes of the invention.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, comprising:
inputting into a health model, by the computing device, a plurality of health data and sensor data collected for an individual for a predetermined time period;
determining, by the computing device, a probability of an onset of at least one symptom of whole body vibration syndrome for the individual from the plurality of health data and sensor data; and
sending, by the computing device, a prediction of the onset of the at least one symptom of whole body vibration syndrome to a user device for display on the user device.

2. The method of claim 1, further comprising training the health model to predict the onset of the at least one symptom of whole body vibration syndrome.

3. The method claim 2, wherein the training the health model comprises applying a time-to-event analysis of a collection of health data and sensor data for a plurality of individuals for the predetermined period of time to generate an initial model represented by a function S(t)=P(T>t), wherein T is a random variable denoting time before any symptom develops in any individual.

4. The method of claim 1, further comprising applying a time-to-event analysis to analyze the plurality of health data and the sensor data to determine the probability of the onset of symptoms of whole body vibration syndrome.

5. The method of claim 1, further comprising determining a ranked list of a plurality of covariates among the plurality of health data and sensor data influences a development of the at least one symptom of whole body vibration syndrome.

6. The method of claim 1, wherein the sensor data comprises vibration exposure.

7. The method of claim 1, wherein the sensor data comprises vibration intensity.

8. The method of claim 1, wherein the sensor data comprises acoustic noise.

9. The method of claim 1, wherein:
the sensor data collected for the individual is collected by a sensory system operably coupled to a plurality of Internet of Things (IoT) sensor devices; and
the sending the prediction includes sending a recommendation to change a route of travel by the user.

10. The method of claim 1, wherein the user device is the user device of a health care provider.

11. The method of claim 1, wherein the prediction is estimated at time t by a quotient of individuals not developing any symptom of whole body vibration syndrome beyond time t divided by a total number of the individuals included in training the health model.

12. A computer program product comprising one or more computer readable storage media having program instructions collectively stored on the one or more computer readable storage media, the program instructions executable to:
receive, by a computing device, a collection of health data and sensor data for a plurality of individuals for a predetermined period of time;
train, by the computing device, a health model from the collection of the health data and sensor data;
store, by the computing device, the trained health model on the one or more computer readable storage media; and
determine, by the computing device, a probability of the onset of at least one symptom of whole body vibration syndrome for an individual from the trained health model.

13. The computer program product of claim 12, wherein the executable instructions are further executable to apply, by the computing device, a health analysis to analyze the plurality of health data and the sensor data collected for the individual to determine the probability of the onset of symptoms of whole body vibration syndrome.

14. The computer program product of claim 12, wherein the executable instructions are further executable to send, by the computing device, a prediction of the onset of the at least one symptom of whole body vibration syndrome to a user device for display on the user device.

15. The computer program product of claim 14, wherein the prediction is estimated at time t by a quotient of the plurality of individuals not developing any symptom of whole body vibration syndrome beyond time t divided by a total number of the plurality of individuals.

16. The computer program product of claim 12, wherein the health model accounts for censoring in which at least one of the plurality of individuals does not experience at least one symptom of whole body vibration syndrome during the predetermined period of time.

17. The computer program product of claim 12, wherein the executable instructions are further executable to apply a time-to-event analysis to generate an initial model represented by a function S(t)=P(T>t), wherein T is a random variable denoting time before any symptom develops in any individual.

18. The computer program product of claim 12, wherein the sensor data collected for the plurality of individuals is collected by a sensory system operably coupled to a plurality of Internet of Things (IoT) sensor devices.

19. The computer program product of claim 18, wherein the sensor data collected includes vibration exposure, ambient temperature, and acoustic noise.

20. A system comprising:
a processor, a computer readable memory, one or more computer readable storage media, and program instructions collectively stored on the one or more computer readable storage media, the program instructions executable to:
receive, by a computing device, a plurality of health data and sensor data collected for an individual for a predetermined time period as a plurality of parameters for input into a health model to predict an onset of at least one symptom of whole body vibration syndrome for the individual;
apply, by the computing device, a time-to-event analysis of the health model to analyze the plurality of health data and the sensor data collected for the individual for the predetermined time period to determine the probability of the onset of symptoms of whole body vibration syndrome;
determine, by the computing device, a probability of the onset of the at least one symptom of whole body vibration syndrome estimated at time t by a quotient of a plurality of individuals used to train the health model who did not develop any symptom of whole body vibration syndrome beyond time t divided by the total number of the plurality of individuals; and
send, by the computing device, a prediction of the onset of the at least one symptom of whole body vibration syndrome to a user device for display on the user device.
